SEQUENCE LISTING (11 sequences)

SEQ ID NO: 1 - protein, 530 aa, Oryctolagus cuniculus
SKSHSEAGSAFIQTQQLHAAMADTFLEHMCRLDIDSAPITARNTGIICTIGPASRSVETLKEMIKSGMNVARMNFSHGTHEYHAETIKNVRTATESFASDPILYRPVAVALDTKGPEIRTGLIKGSGTAEVELKKGATLKITLDNAYMEKCDENILWLDYKNICKVVDVGSKVYVDDGLISLQVKQKGPDFLVTEVENGGFLGSKKGVNLPGAAVDLPAVSEKDIQDLKFGVDEDVDMVFASFIRKAADVHEVRKILGEKGKNIKIISKIENHEGVRRFDEILEASDGIMVARGDLGIEIPAEKVFLAQKMIIGRCNRAGKPVICATQMLESMIKKPRPTRAEGSDVANAVLDGADCIMLSGETAKGDYPLEAVRMQHLIAREAEAAMFHRKLFEELARSSSHSTDLMEAMAMGSVEASYKCLAAALIVLTESGRSAHQVARYRPRAPIIAVTRNHQTARQAHLYRGIFPVVCKDPVQEAWAEDVDLRVNLAMNVGKARGFFKKGDVVIVLTGWRPGSGFTNTMRVVPVP

SEQ ID NO: 2 - protein, 4 aa, Artificial Sequence
  Description of Artificial Sequence: Synthetic peptide
AVGL

SEQ ID NO: 3 - protein, 6 aa, Artificial Sequence
  Description of Artificial Sequence: Synthetic peptide
EKIKLI

SEQ ID NO: 4 - protein, 5 aa, Artificial Sequence
  Description of Artificial Sequence: Synthetic peptide
WLHYI

SEQ ID NO: 5 - protein, 5 aa, Artificial Sequence
  Description of Artificial Sequence: Synthetic peptide
RSSFG

SEQ ID NO: 6 - protein, 6 aa, Artificial Sequence
  Description of Artificial Sequence: Synthetic peptide
RTEILK

SEQ ID NO: 7 - protein, 7 aa, Artificial Sequence
  Description of Artificial Sequence: Synthetic peptide
PKILPWR

SEQ ID NO: 8 - protein, 5 aa, Artificial Sequence
  Description of Artificial Sequence: Synthetic peptide
ERTKI

SEQ ID NO: 9 - protein, 8 aa, Artificial Sequence
  Description of Artificial Sequence: Synthetic peptide
WYHPLLKK

SEQ ID NO: 10 - protein, 11 aa, Artificial Sequence
  Description of Artificial Sequence: Synthetic peptide
RTGFSSGRTLL

SEQ ID NO: 11 - protein, 4 aa, Artificial Sequence
  Description of Artificial Sequence: Synthetic peptide
PKIL

(19) United States
(12) Patent Application Publication
(10) Pub. No.: US 2005/0074816 A1
(43) Pub. Date: Apr. 7, 2005

(54) Method for protein identification from tandem mass spectral employing both spectrum comparison and de novo sequencing for biomedical applications

(76) Inventors: Mark W. Duncan, Denver, CO (US); Srdjan Askovic, Salt Lake City, UT (US); Kim Fung, New South Wales (AU)

Correspondence Address: PATTON BOGGS, L.L.P., 2001 ROSS AVENUE, SUITE 3000, DALLAS, TX, 75201, US

(21) Appl. No.: 10/886,779

(22) Filed: Jul. 7, 2004

Related U.S. Application Data
(60) Provisional application No. 60/485,633, filed on Jul. 7, 2003.
(60) Provisional application No. 60/485,476, filed on Jul. 7, 2003.
(60) Provisional application No. 60/485,632, filed on Jul. 7, 2003.

(51) Int. Cl. G01N 33/53; G06F 19/00; G01N 33/48; G01N 33/50

(52) U.S. Cl. 435/7.1; 702/19

(57) ABSTRACT

A method and algorithm for identifying a protein sequence from mass spectral data combines peptide spectrum matching analysis or spectrum comparison approaches and de novo sequencing approaches. The algorithm of the invention identifies peptide sequences determined independently by each approach, then compares the results and assigns a score reflecting the “goodness” of the match with a full-length protein sequence. Because peptides are identified using independent approaches, the probability of a correct match is increased.

### CROSS-REFERENCE TO RELATED APPLICATIONS

This application claims the benefit of and priority to U.S. Provisional Application No. 60/485,633, filed Jul. 7, 2003, which is hereby incorporated by reference.

This application also incorporates by reference commonly-owned U.S. Provisional Application Nos. 60/485,476 and 60/485,632, both filed on Jul. 7, 2003.

### BACKGROUND OF THE INVENTION

1. Field of the Invention

The present application relates to a method for protein identification from tandem mass spectral employing both spectrum comparison and de novo sequencing for biomedical applications.

2. Description of Related Art

Improvements in mass spectrometry, the availability of larger, more complete nucleic acid and protein databases, and an increase in affordable computing power have fueled the development of proteomics. Data analysis, however, is currently the bottleneck in the whole process of protein identification.

There are two main experimental approaches to protein identification, both based on mass spectrometry, that form the basis of current proteomics methods:

Peptide mass fingerprinting (PMF): In peptide mass fingerprinting, proteins are first digested by an enzyme, and the masses of the resulting peptides (i.e., the peptide mass fingerprint) is generated by mass spectrometry. The spectrum is then compared to the predicted “fingerprint”, or pattern, for all proteins in the database. PMF relies on relatively pure samples (if more than two proteins are in the mixture, identification becomes very difficult).

MS/MS analysis: Here a protein is digested to peptides, and then selected peptides are analyzed in the tandem mass spectrometer. Tandem mass spectrometry is a very powerful method for protein identification because the information content of a MS/MS spectrum is high and the fragments formed are sequence-dependent. The interpretation of the MS/MS spectrum is, however, slow and error prone. Currently the experimentally determined fragments can yield a sequence, or partial sequence, by one of two alternative approaches—

Spectrum Analysis (or spectrum comparison): The experimentally determined fragments in a MS/MS spectrum are compared to the predicted fragments generated in silico for each peptide entry in the database of the same mass (within a predetermined error). There are problems, however, inherent to the spectrum matching process including a high rate of false positives and the inability to identify peptide if the sequence is not in the database.

De Novo Analysis: Mass differences between peaks in a MS/MS spectrum can be used to infer the amino acid sequence of a peptide. De novo sequencing depends on the presence of a near complete fragment ion series, and any interruption in the series will cause difficulties in interpretation. As a result, de novo sequencing cannot always be used as a standalone method to identify peptides. The complete peptide sequence can seldom be determined with a high degree of accuracy because the fragmentation pattern is frequently incomplete, and interpretation can be complex. However, even in the case when a complete peptide sequence cannot be identified de novo, there is usually enough information to determine short sequence tags (or a partial sequence).

Spectrum matching and de novo sequencing are completely different approaches offering their own strengths and weaknesses. Currently no algorithm employs both strategies to determine a peptide sequence from a MS/MS platform.

### BRIEF DESCRIPTION OF THE DRAWINGS

FIG. 1 is a conversion of LCQ RAW files into a peak list. The first step done by Wombat.pl is to extract the peak list (.dta files) from the binary LCQ .raw files. This is done by executing a system call to the lcq_dta.exe program.

FIG. 2 is a main program flow. Each peak list is passed first to the de-noising module, which returns a modified peak list. The numbers of peaks returned varies from 100 to 40. This list is then used by the spectrum comparison module, as well as by the de novo sequencing module. Results from both are saved, and the de-noising module is called again and a new peak list (10 peaks fewer) is returned. The process is repeated until the final peak list is between 40 and 50 entries. After the last iteration, the best matches are chosen. The top matches from the spectrum comparison (currently set at 200) are compared to sequences or sequence tags from the de novo search and the top scoring peptides (currently set at 50) are saved to the final results file.

FIG. 3 is a protein assembly. The top scoring peptides from the final list (providing their scores are above the pre-set cutoff) are taken from each and peptides are sorted based on the gi number they are found in, so that the ones occurring in the same proteins are together. (Currently the top 5 peptides are used.) Total and relative protein scores are then calculated based on the peptide scores, protein size, and sequence coverage.

FIG. 4 is an example of the screen output from Wombat.

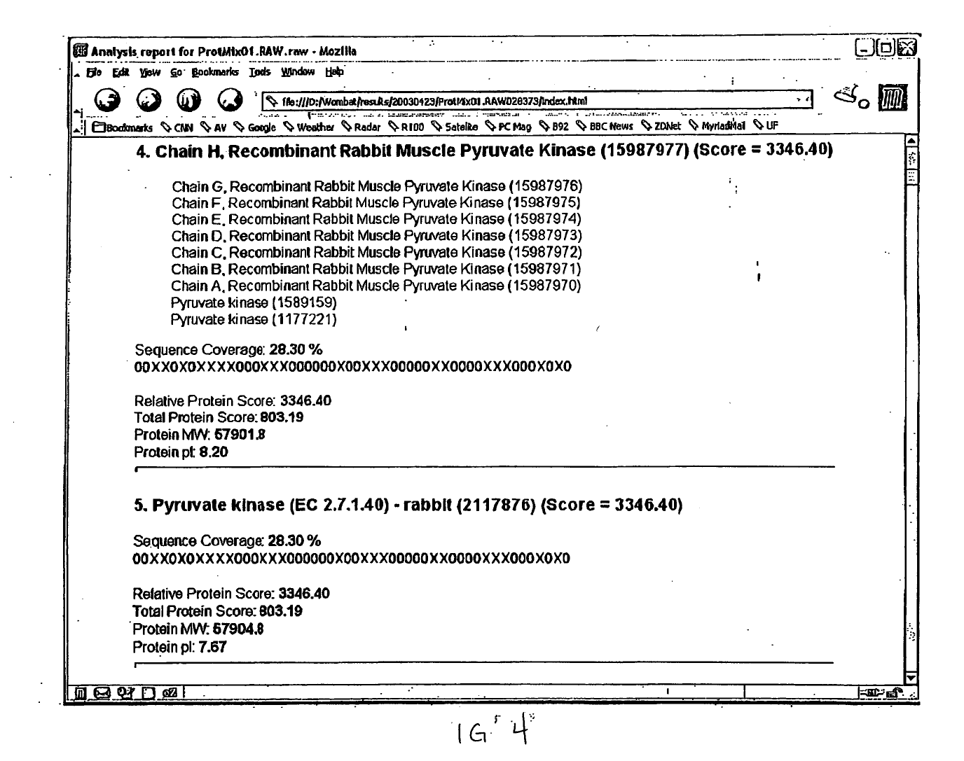

FIG. 5 is an example of the screen output from Wombat.

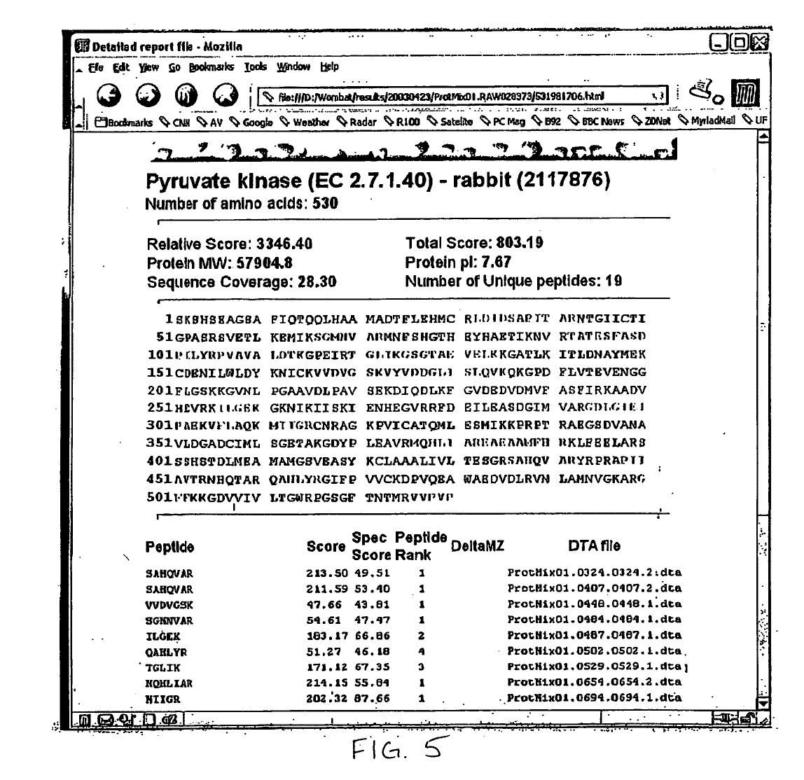

### DETAILED DESCRIPTION OF THE PREFERRED EMBODIMENT

In the following detailed description of the preferred embodiments, reference is made to the accompanying drawings, which form a part hereof and in which is shown by way of illustration specific preferred embodiments in which the invention may be practiced. These embodiments are described in sufficient detail to enable those skilled in the art to practice the invention, and it is understood that other embodiments may be utilized and that logical software, electrical, mechanical, structural, and chemical changes may be made without departing from the spirit or scope of the invention. To avoid detail not necessary to enable those skilled in the art to practice the invention, the description may omit certain information known to those skilled in the art. The following detailed description is, therefore, not to be taken in a limiting sense, and the scope of the present invention is defined only by the appended claims.

The software program of the present invention (“Wombat”) combines spectrum matching and de novo sequencing approaches to determine peptide sequences from the MS/MS spectra. Wombat combines both approaches and identifies sequences by each method independently, then compares the results and assigns a score. The two algorithms that form the basis of Wombat—spectrum comparison and de novo interpretation—were independently developed and tested extensively as separate modules. Final adjustment of the algorithms was done empirically, by analyzing manually verified peak lists. Wombat then combines all the information to determine the list of the most likely protein precursors of each peptide and assigns a protein score (i.e., an indication of the “goodness” of the match). Proteins are then sorted based on their score and a result page containing coverage information, scores, links to the original peak lists, and protein entries in GenBank is generated.

Because the identification of peptides is done using two independent approaches, the probability of identifying incorrect peptides is dramatically reduced. Scores for the peptides are increased dramatically if de novo have matched the exact same peptide sequence as found by spectrum matching, and to a lesser degree if shorter sequence tag was found by analyzing spectrum. Only then, peptides are sorted based on the protein they matched, and total protein score is calculated.

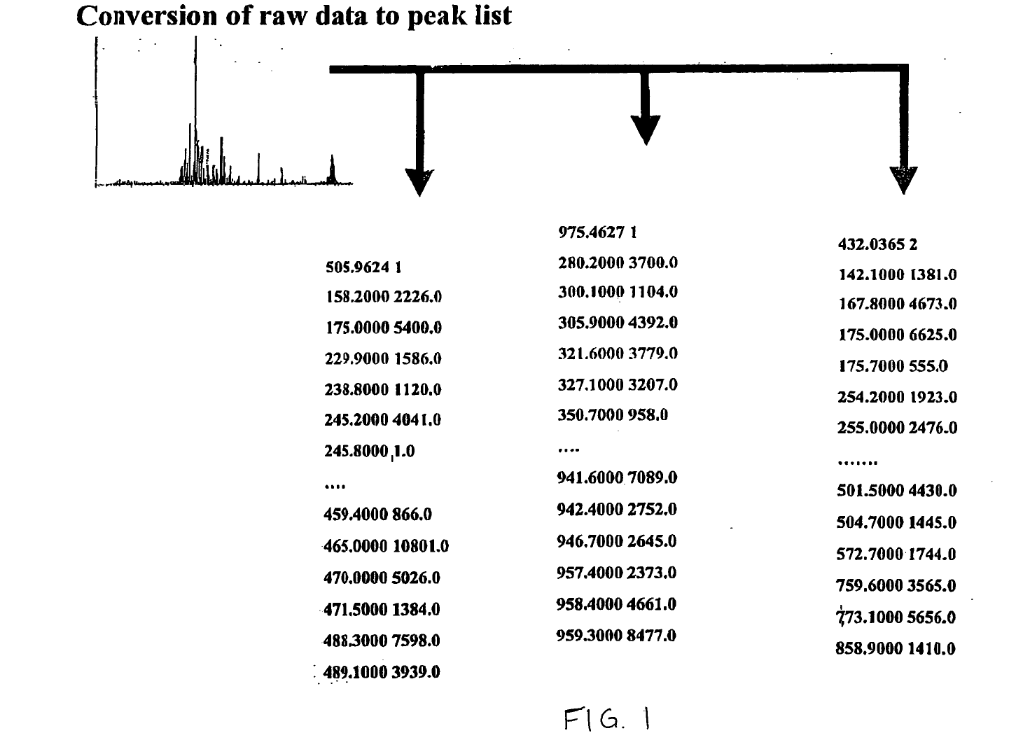

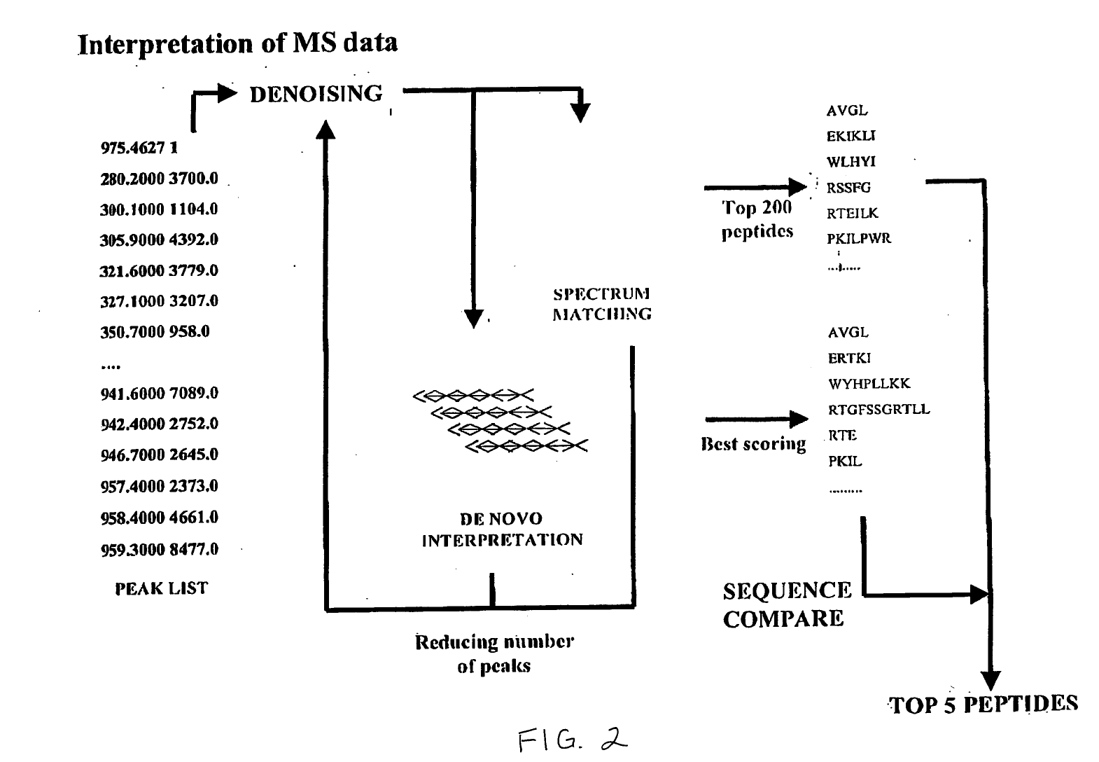

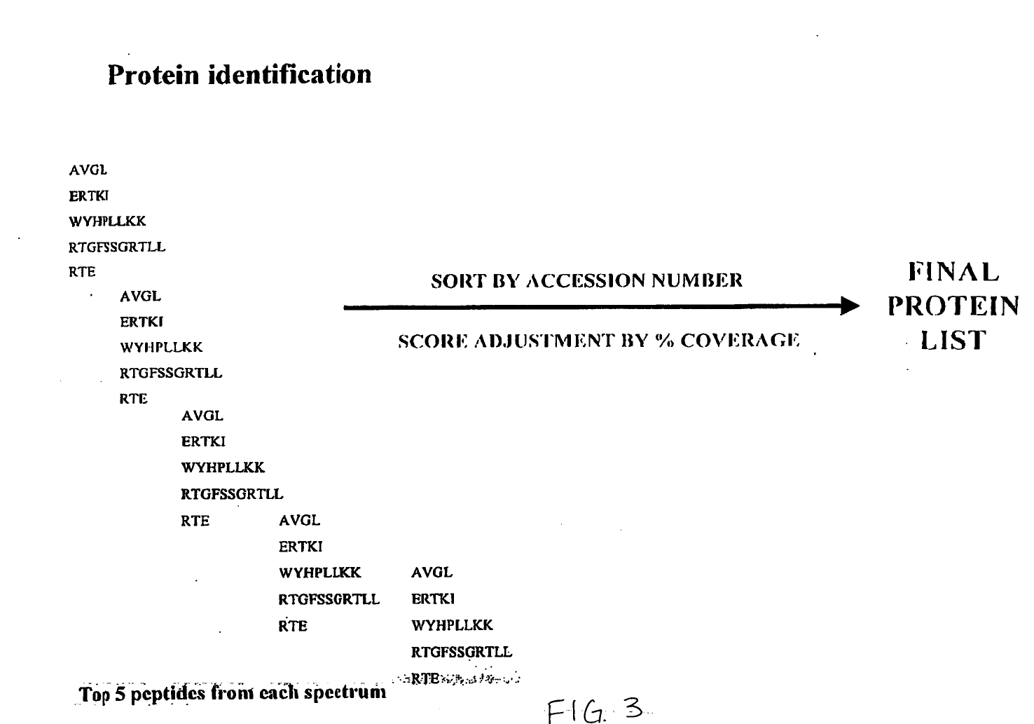

The Wombat program consists of several modules. Two perl scripts are used to execute the Wombat program: Wombat.pl (FIGS. 1 and 2) and assemble.pl (FIG. 3). Wombat.pl is a perl wrapper that manages several program modules. Wombat.pl extracts the peak lists (.dta files) from LCQ .raw files (or similar) into a temporary directory (FIG. 1). Each .dta file is then processed independently (FIG. 2). The index.exe module (written in C++) is called with the precursor mass and the peptide mass tolerance. Index.exe creates the list of all peptide from the database, which are in the mass range. Then the peak list from the .dta file is run through the denoiseme.pl module (written in perl), which returns the de noised peak list. The denoiseme.pl module passes the .dta filename and the maximum number of peaks it should return (that number varies from 100 to 40). Next, the peptide list, and de noised peak list are passed to the smatching.exe module (written in C++). Smatching.exe module predicts the fragments for each peptide from the list, and compares that to the actual observer spectrum, creating the list of the top 200 peptide candidates. The same peak list is also passed to the de novo module (denovo.pl; written in perl), which determines either the complete peptide sequence (if possible), or the sequence tags. The smatching.exe and denovo.pl modules run several times with a decreasing number (10) of peaks on the each consecutive run. When the number of peaks reaches 40, the best scoring peptides for each module are determined (this is handled by Wombat.pl). The top 200 peptides from the smatching.exe module are then passed to the next module (seqcompare.exe—written in C++) together with the sequence list from the de novo module. The seqcompare.exe module matches the sequences obtained by the de novo module against the top 200 peptide list reported by the sequence matching module, and if a match is found, it adjusts the peptide scores. Upon completion, the list of the top 200 peptide is sorted again according to score, and the top 50 peptides are written to the final results file. The final results file is then processed by the next module (assemble.pl—written in perl—FIG. 3), which takes the top 5 peptides for each .dta file, assembles them, and sorts them by their gi number (i.e., so that those belonging to the same protein are together). The final protein score is based on the peptide scores for each of the peptides that matched that protein, and on the total sequence coverage. We have empirically determined that protein scores of 50 or more are very likely to be correct, and these are therefore designed as definitive matches; proteins with scores of 20-50 are possibly correct and are designated as possible matches. Proteins with a score of <20 are not likely to be correct and are therefore designated as unlikely matches. The results report is generated in HTML format.

The main results page contains matched protein names, hyperlinked to the complete results for that protein. Also listed are the alternate names for that exact sequence and the corresponding gi numbers. The sequence coverage, the relative and total protein scores, the protein MW and pI are also reported. Each identified peptide is listed together with its complete score (score), spectrum matching score (spec score), the rank in the final peptide list as well as with the name of the .dta file it came from.

The function of each module is summarized in the sections that follow.

Database indexing: A separate module is used to pre-index the sequence databases based on the selected enzyme and the number of missed cleavage sites. (This module is written in C++). The indexed database allows fast peptide access based on mass (i.e., peptides that have their mass in the certain range can be extracted quickly). If no enzyme is selected then the peptide lists are generated “on the fly” from the flat fasta file, a process which takes much longer.

De noising: Both de novo and spectrum matching are performed several times on each peak list, and the highest scoring results are used in further comparisons. Each spectrum is first filtered for noise (peaks that are below 500 counts are removed), peaks are de-isotoped, and then sorted based on intensity (i.e., from high to low). In the first iteration, the top 100 peaks are used. (If there are less than 100 peaks in the spectrum then all are employed.) For each iteration, 10 fewer peaks are used (i.e., the 10 peaks with the lowest intensity are dropped), until a minimum of 40 peaks are employed.

Spectrum analysis: MS/MS peak lists are extracted from LCQ raw files using lcq_dta.exe utility (with the following parameters: “-A -G1 -I20 -B400 -T4000”). The general strategy, however, is not restricted to employing LCQ data. Each peak list (.dta file) is independently analyzed. The list of the peptides in the mass range (+/− peptide mass tolerance) is extracted from the pre-indexed peptide database (enzyme digested peptide database) and for each peptide from this list the predicted MS/MS fragments are calculated (m/z for 1+, and 2+ if necessary, of y, b, a and the neutral loss fragments of y, b and a ions). The observed peak list is then compared to the predicted m/z lists for each peptide from the database. The score for each peptide is determined and the peptides are sorted based on their score.

De novo Analysis: This part of the algorithm operates independently of Spectrum Analysis on the same observed peak list. The de novo module determines sequence tags based on the following:
+1 and +2 y ions read from the highest m/z values,
+1 and +2 b ions read from the highest mass values,
+1 and +2 y ions read from the low mass values (i.e., from 250),
+1 and +2 b ions read from the lowest m/z values (i.e., 250),
+1 and +2 read from the highest intensity peak in both directions (i.e., without assuming y or b).

Finally, a bi-directional sequence is identified based on the b ions (+1 and +2). Each sequence tag is assigned a score. Sequences from the y and b ions are combined to determine the complete peptide sequence (i.e., within the given precursor mass range +/− the peptide tolerance).

Integration of spectrum analyses and de novo analysis: The highest scoring 200 peptides from spectrum analysis are compared to the sequences and sequence tags from the de novo analysis. If there is good agreement between the two, the score for the peptide is increased based on the score of the sequence or a sequence tag that it matched. The 200-peptide list is then re-sorted by score, and the top 50 peptides are written to the final result file.

Protein Identification: For protein analysis the top scoring peptides are used (only if they have score higher than the empirically determined significance cutoff). After all peak lists contained in the original .raw file have been analyzed, another module was used to determine the precursor proteins. This is done by initially sorting the target peptide list according to gi-number. (This information is also obtained from the pre-indexed file.) Protein scores are calculated based on the peptide scores for each constituent peptide and the total sequence coverage.

Wombat combines spectrum matching and de novo interpretation of tandem mass spectral data to identify proteins. Because the algorithm incorporates de novo sequencing and spectrum matching, this increases the certainty of protein identifications. The ambiguity associated with non-significant peptide matches is removed and as a result, the potential for false positive results are reduced. Since spectrum matching and de novo sequencing are independent approaches to sequence assignment, when both yield similar or identical information, the accuracy of assignment is markedly enhanced. We have demonstrated that the combination of de novo sequencing and spectrum matching provide more accurate results than either method employed alone. When compared to existing (commercial) approaches, e.g., Mascot and Sequest, our approach provides high sequence coverage and most importantly, returns fewer false positives and false negative results.

As will be recognized by those skilled in the art, the innovative concepts described in the present application can be modified and varied over a tremendous range of applications, and accordingly the scope of patented subject matter is not limited by any of the specific exemplary teachings given.

While the invention has been particularly shown and described with reference to a preferred embodiment, it will be understood by those skilled in the art that various changes in form and detail may be made therein without departing from the spirit and scope of the invention.

None of the description in the present application should be read as implying that any particular element, step, or function is an essential element which must be included in the claim scope: THE SCOPE OF PATENTED SUBJECT MATTER IS DEFINED ONLY BY THE ALLOWED CLAIMS. Moreover, none of these claims are intended to invoke paragraph six of 35 USC § 112 unless the exact words “means' for” are followed by a participle.

1. A method for identifying proteins from mass spectral data comprising the steps of:
determining a first set of peptide sequences using spectrum matching techniques;
determining a second set of peptide sequences using de novo sequencing techniques;
comparing the first set of peptide sequences to the second set of peptide sequences; and
assigning a score to each peptide sequence based at least in part on whether the peptide sequence was present in both the first set and the second set.
